(19) United States
(12) Patent Application Publication
(10) Pub. No.: US 2023/0256007 A1
(43) Pub. Date: Aug. 17, 2023

(54) COMPOSITION FOR IMPROVING SEQUELAE OF VIRAL INFECTION AND/OR REDUCING WORSENING OF SYMPTOMS

(71) Applicant: MiZ Company Limited, Kamakura-shi (JP)

(72) Inventors: Fumitake SATOH, Kamakura-shi (JP); Yoshiyasu TAKEFUJI, Fujisawa (JP); Takashi YAMAMURA, Tokyo (JP); Bunpei SATO, Kamakura-shi (JP); Shinichi HIRANO, Kamakura-shi (JP); Yusuke ICHIKAWA, Kamakura-shi (JP)

(73) Assignee: MiZ Company Limited, Kamakura-shi (JP)

(21) Appl. No.: 18/100,148

(22) Filed: Jan. 23, 2023

(30) Foreign Application Priority Data
Jan. 24, 2022 (JP) JP 2022-19103

(51) Int. Cl. A61K 33/00; A61K 9/00; A61P 31/16

(52) U.S. Cl. CPC A61K 33/00; A61K 9/0073; A61P 31/16

(57) ABSTRACT

Provided is a novel composition that has no side effects and can be easily produced, for use in improving symptoms of sequelae of viral infection. A composition comprising a hydrogen gas-containing gas as an active ingredient, for use in improving symptoms of sequelae of viral infection, such as pulmonary fibrosis, feeling of fatigue, malaise, shortness of breath, difficulty breathing, and/or decrease in activity levels associated with these symptoms, in a patient with sequelae of viral infection, and/or a method for improving symptoms, such as decrease in activity levels associated with these symptoms.

### RELATED APPLICATIONS

The present patent document claims the benefit of priority to Japanese Patent Application No. JP 2022-19103, filed Jan. 24, 2022, the entire contents of which are incorporated herein by reference.

### BACKGROUND OF THE INVENTION

### 1. Field of the Invention

The present invention relates to a composition for improving symptoms of sequelae of viral infection and/or reducing worsening of the symptoms, such as fatigue, headache, body pain, decreased thinking power, loss of concentration, and/or decrease in activity levels associated with these symptoms.

The present invention also relates to a method for improving sequelae of viral infection and/or reducing worsening of their symptoms.

### 2. Description of the Related Art

Sequelae of viral infection, such as influenza virus infection or novel coronavirus infection (COVID-19), include severe general malaise and appear as various symptoms, such as prolonged fatigue, extreme malaise aggravated after exertion, low-grade fever, headache, muscle pain, faintness, cognitive impairment, restless sleep, and lightheadedness.

The main symptom of sequelae of viral infection, such as influenza virus infection or novel coronavirus infection (COVID-19), is whole body pain of unknown cause. Pain shows symptoms of enthesitis and severe pain that occurs in muscles, joints, etc., and spreads from the trunk and extremities to the entire body.

Because all of the symptoms of sequelae of viral infection, such as influenza virus infection or novel coronavirus infection (COVID-19), are indescribable, even if patients recover from infection with such viruses, they will sometimes be unable to engage in healthy social activities. However, the pathological mechanisms of sequelae of viral infection, such as influenza virus infection or novel coronavirus infection (COVID-19), are unknown, and there is no cure.

In contrast, hydrogen, which is an active ingredient of the present invention, has antioxidant reactivity that suppresses oxidative stress caused by active oxygen species, and there is a precedent for improving chronic bronchitis and obstructive pulmonary bronchitis in studies using mice (Japanese Patent No. 6628449). However, there is no precedent for conforming the effect of improving sequelae of viral infection by hydrogen.

An object of the present invention is to provide a novel composition that has no side effects and can be easily produced, for use in improving symptoms of sequelae of viral infection and/or reducing worsening of the symptoms.

In recent years, it has been proposed that hydrogen molecules may be effective against pneumonia by scavenging hydroxyl radicals generated inside the mitochondria of cells, thereby reducing pneumonia (Japanese Patent No. 6628449). However, as for symptoms of sequelae of viral infection, there have been no reports on whether inhalation, sucking, or drinking of a hydrogen gas-containing gas can improve the symptoms of sequelae of viral infection and/or suppress worsening of the symptoms.

### SUMMARY OF THE INVENTION

As a result of extensive research, the present inventors unexpectedly found that a hydrogen gas-containing gas improves specific symptoms of sequelae of viral infection and/or suppresses worsening of the symptoms.

Therefore, the present invention includes the following characteristics.

(1) A composition comprising a hydrogen gas-containing gas as an active ingredient, for use in improving symptoms of sequelae of viral infection and/or reducing worsening of the symptoms in a patient with sequelae of viral infection.

(2) The composition according to (1), wherein the virus of the viral infection is one or more viruses selected from the group consisting of coronavirus, novel coronavirus, influenza virus, norovirus, cytomegalovirus, yellow fever virus, herpes virus, measles virus, rubella virus, chickenpox virus, Ebola virus, Marburg virus, adenovirus, papillomavirus, immunodeficiency virus, hepatitis B virus, hantavirus, and variants of these viruses.

(3) The composition according to (1) or (2), wherein the symptoms of sequelae of the viral infection include at least pulmonary fibrosis, feeling of fatigue, malaise, shortness of breath, difficulty breathing, palpitation, muscle weakness, low-grade fever, muscle pain, decreased sensation, joint pain, chest discomfort, chest pain, sore throat, headache, heavy head, body pain, generalized pain, faintness, cognitive impairment, memory impairment, language retrieval difficulty, gastrointestinal disorder, abdominal pain, loss of concentration, dizziness when standing up, fragility, irregular body temperature, chills, hair loss, cold hands and feet, heat and/or cold intolerance, sweating, hot flush, lymph node tenderness, significant changes in body weight, tinnitus, confusion, sleeping disorder, lightheadedness, dizziness, anorexia, feeling of faintness, bradycardia, blood pressure drop, nausea, prolonged cough, brain fog, rash, and/or decrease in activity levels associated with these symptoms.

(4) The composition according to any one of (1) to (3), wherein the hydrogen gas-containing gas has a hydrogen concentration of more than zero (0) and 18.5 volume% or less.

(5) The composition according to any one of (1) to (4), wherein the composition is administered by inhalation to the patient.

(6) A method for producing the composition according to any one of (1) to (5), comprising using a hydrogen gas generator, thereby producing the composition.

### EFFECTS OF INVENTION

The present invention makes it possible to, by inhalation, sucking, or the like of hydrogen gas, significantly improve symptoms caused by sequelae of viral infection and/or suppress worsening of the symptoms, including at least pulmonary fibrosis, feeling of fatigue, malaise, shortness of breath, difficulty breathing, palpitation, muscle weakness, low-grade fever, muscle pain, decreased sensation, joint pain, chest discomfort, chest pain, sore throat, headache, heavy head, body pain, generalized pain, faintness, cognitive impairment, memory impairment, language retrieval difficulty, gastrointestinal disorder, abdominal pain, loss of concentration, dizziness when standing up, fragility, irregular body temperature, chills, hair loss, cold hands and feet, heat and/or cold intolerance, sweating, hot flush, lymph node tenderness, significant changes in body weight, tinnitus, confusion, sleeping disorder, lightheadedness, dizziness, anorexia, feeling of faintness, bradycardia, blood pressure drop, nausea, prolonged cough, brain fog, rash, and/or decrease in activity levels associated with these symptoms.

### DETAILED DESCRIPTION OF THE PREFERRED EMBODIMENTS

The present invention will be described in more detail below.

### 1. Sequelae of Viral Infection and Symptoms Thereof

The patients with viral infection in the present invention refer to patients infected with pathogenic viruses, such as coronavirus including the novel coronavirus that causes COVID-19, influenza virus, norovirus, cytomegalovirus, yellow fever virus, herpes virus, measles virus, rubella virus, chickenpox virus, Ebola virus, Marburg virus, adenovirus, papillomavirus, immunodeficiency virus, hepatitis B virus, and hantavirus. Further, sequelae of viral infection refer to symptoms that remain even after viral infection caused by infection with these viruses is cured. The sequelae of viral infection in the present invention are clearly distinguished from symptoms that develop during infection with viruses. Therefore, the patients with sequelae of viral infection in the present invention refer to patients for which virus tests, such as PCR, have confirmed viral infection, but the virus has been mostly eliminated from the body afterwards.

However, in the case of retroviruses, such as immunodeficiency virus and papillomavirus, the sequelae of viral infection include those of a condition in which although symptoms of viral infection have subsided due to suppression of virus expression after the symptoms of viral infection develop due to virus expression, and the retrovirus gene has been incorporated into host nucleic acids, such as chromosomes. In addition, the sequelae of viral infection in the present invention include sequelae of a condition in which herpes virus and like viruses are not completely eliminated and the viruses remain latent in the body after viral infection develops and is cured.

Sequelae of viral infection are symptoms that appear after infection with various pathogenic viruses, and examples of such symptoms include pulmonary fibrosis, feeling of fatigue, malaise, shortness of breath, difficulty breathing, palpitation, muscle weakness, low-grade fever, muscle pain, decreased sensation, joint pain, chest discomfort, chest pain, sore throat, headache, heavy head, body pain, generalized pain, faintness, cognitive impairment, memory impairment, language retrieval difficulty, gastrointestinal disorder, abdominal pain, loss of concentration, dizziness when standing up, fragility, irregular body temperature, chills, hair loss, cold hands and feet, heat and/or cold intolerance, sweating, hot flush, lymph node tenderness, significant changes in body weight, tinnitus, confusion, sleeping disorder, lightheadedness, dizziness, anorexia, feeling of faintness, bradycardia, blood pressure drop, nausea, prolonged cough, brain fog, rash, and/or decrease in activity levels associated with these symptoms.

Examples of viruses related to sequelae of viral infection in the present invention include pathogenic viruses, such as coronavirus, including the novel coronavirus that causes COVID-19, influenza virus, norovirus, cytomegalovirus, yellow fever virus, herpes virus, measles virus, rubella virus, chickenpox virus, Ebola virus, Marburg virus, adenovirus, papillomavirus, immunodeficiency virus, hepatitis B virus, and hantavirus.

For sequelae of viral infection, no abnormalities are often confirmed by examination even though patients complain of physical symptoms. Occasionally, symptoms, such as indescribable pain, fatigue that is qualitatively different from overwork, decreased thinking power, and abnormal hypersensitivity, may occur.

### 2. Composition for Improving Sequelae of Viral Infection and/or Reducing Worsening of Symptoms

A first aspect of the present invention provides a composition comprising a hydrogen gas-containing gas as an active ingredient, for use in improving symptoms caused by sequelae of viral infection and/or reducing worsening of the symptoms in a patient with sequelae of viral infection, the symptoms including at least pulmonary fibrosis, feeling of fatigue, malaise, shortness of breath, difficulty breathing, palpitation, muscle weakness, low-grade fever, muscle pain, decreased sensation, joint pain, chest discomfort, chest pain, sore throat, headache, heavy head, body pain, generalized pain, faintness, cognitive impairment, memory impairment, language retrieval difficulty, gastrointestinal disorder, abdominal pain, loss of concentration, dizziness when standing up, fragility, irregular body temperature, chills, hair loss, cold hands and feet, heat and/or cold intolerance, sweating, hot flush, lymph node tenderness, significant changes in body weight, tinnitus, confusion, sleeping disorder, lightheadedness, dizziness, anorexia, feeling of faintness, bradycardia, blood pressure drop, nausea, prolonged cough, brain fog, rash, and/or decrease in activity levels associated with these symptoms.

In the present specification, “hydrogen,” which is an active ingredient of the composition of the present invention, is molecular hydrogen (i.e., gaseous hydrogen), and is simply referred to as “hydrogen” or “hydrogen gas” unless otherwise specified. Further, the term “hydrogen” used in the present specification refers to hydrogen represented by the molecular formula of H₂, D₂ (deuterium), or HD (hydrogen deuteride), or a gas mixture thereof. D₂ is expensive, but is known to have a stronger superoxide scavenging effect than H₂. The type of hydrogen usable in the present invention is H₂, D₂ (deuterium), HD (hydrogen deuteride), or a gas mixture thereof, and preferably H₂. Alternatively, D₂ and/or HD may be used in place of H₂, or as a mixture with H₂.

The hydrogen gas-containing gas is preferably air containing hydrogen gas, or a gas mixture containing hydrogen gas and oxygen gas. The concentration of hydrogen gas in the hydrogen gas-containing gas is more than zero (0) and 18.5 volume% or less, preferably 10 volume% or less, for example, 0.1 to 18.5 volume%, preferably 1 to 18.5 volume%, for example, 2 to 18.5 volume%, 3 to 18.5 volume%, 3 to 18.5 volume%, 4 to 18.5 volume%, 5 to 18.5 volume%, 6 to 18.5 volume%, 7 to 18.5 volume%, or 9 to 18.5 volume%. In the present invention, the effect of improving symptoms of sequelae of viral infection and/or reducing worsening of the symptoms tends to be enhanced as the hydrogen gas concentration increases at a level equal to or less than the explosion limit (18.5 volume% or less, preferably 10 volume% or less).

Since hydrogen is a flammable and explosive gas, in the improvement of symptoms of sequelae of viral infection and/or suppression of worsening of the symptoms, it is preferable to incorporate hydrogen into the composition of the present invention under safe conditions and then administer the composition to a patient with sequelae of viral infection. For example, when the gas other than hydrogen gas is air, the air concentration is within the range of 81.5 to 99.5 volume%, for example.

When the gas other than hydrogen gas is a gas containing oxygen gas, the concentration of oxygen gas is within the range of 21 to 99.5 volume%, for example.

Nitrogen gas can be contained as another main gas. A gas, such as carbon dioxide, which is a gas present in the air, may be contained in an amount equivalent to the abundance thereof in the air.

In the present invention, a hydrogen-dissolved liquid can be administered to or ingested by a patient with sequelae of viral infection in combination with the administration of the hydrogen gas-containing gas, as necessary.

In the case of combined administration with a hydrogen-dissolved liquid, the composition of the present invention can be administered before the administration of the hydrogen-dissolved liquid, simultaneously with the administration of the hydrogen-dissolved liquid, or after the administration of the hydrogen-dissolved liquid.

The hydrogen-dissolved liquid is specifically an aqueous liquid in which hydrogen gas is dissolved. Non-limitative examples of the aqueous liquid include water (e.g., sterile water and purified water), saline, buffer solutions (e.g., buffer solutions having a pH of 4 to 7.4), ethanol-containing water (e.g., ethanol content: 0.1 to 2 volume%), intravenous drips, infusion solutions, injection solutions, beverages, and the like. The hydrogen concentration of the hydrogen-dissolved liquid is, for example, 1 to 10 ppm, for example, 2 to 8 volume%, 3 to 7 volume%, 3 to 6 volume%, 4 to 6 volume%, 4 to 5 volume%, 5 to 10 volume%, 5 to 8 volume%, 6 to 8 volume%, or 6 to 7 volume%, and more preferably 5 to 8 volume%, for example, 6 to 8 volume%, or 6 to 7 volume%. In the present invention, the effect of improving symptoms of sequelae of viral infection and/or reducing worsening of the symptoms tends to be enhanced as the hydrogen gas concentration increases at a level equal to or less than the explosion limit.

A drug for treating sequelae of viral infection may be added to the hydrogen-dissolved liquid. Alternatively, such a drug may be administered separately from the administration of the hydrogen-dissolved liquid or hydrogen gas-containing gas.

The hydrogen gas-containing gas or hydrogen-dissolved liquid is formulated to a predetermined hydrogen gas concentration, and then placed in, for example, a pressure-resistant container (e.g., a stainless-steel cylinder or an aluminum can, preferably a pressure-resistant plastic bottle laminated with an aluminum film on the inside (e.g., a pressure-resistant PET bottle), a plastic bag, an aluminum bag, etc.). Aluminum has the property of being impermeable to hydrogen molecules. Alternatively, the hydrogen gas-containing gas or hydrogen-dissolved liquid may be produced in situ at the time of administration using a device, such as a hydrogen gas generator, a hydrogen water generator, or a hydrogen gas-adding device, for example, a known or commercially available hydrogen gas supply device (a device for generating a hydrogen gas-containing gas), a hydrogen-adding instrument (a device for generating hydrogen water), or a non-destructive hydrogen-adding apparatus (e.g., a device for non-destructively adding hydrogen gas into a bag for a biologically applicable liquid, such as an intravenous drip).

The hydrogen gas supply device makes it possible to mix hydrogen gas generated by the reaction of a hydrogen-generating agent (e.g., metallic aluminum, magnesium hydride, etc.) and water, with a diluting gas (e.g., air, oxygen, etc.) at a predetermined ratio (Japanese Patent No. 5228142, etc.). Alternatively, hydrogen gas generated by using electrolysis of water is mixed with a diluting gas, such as oxygen or air (Japanese Patent No. 5502973, Japanese Patent No. 5900688, etc.). In this way, a hydrogen gas-containing gas having a hydrogen concentration within the range of 0.5 to 18.5 volume% can be prepared.

The hydrogen-adding instrument is a device that generates hydrogen using a hydrogen-generating agent and a pH adjusting agent, and dissolves it in a biologically applicable liquid, such as water (Japanese Patent No. 4756102, Japanese Patent No. 4652479, Japanese Patent No. 4950352, Japanese Patent No. 6159462, Japanese Patent No. 6170605, Japanese Patent Laid-Open No. 2017-104842, Japanese Patent No. 6159462, etc.). The combination of a hydrogen-generating agent and a pH adjusting agent is, for example, a combination of metallic magnesium and a strongly acidic ion-exchange resin or an organic acid (e.g., malic acid, citric acid, etc.), a combination of metallic aluminum powder and calcium hydroxide powder, and the like. In this way, a hydrogen-dissolved liquid with a dissolved hydrogen concentration of about 1 to 10 ppm can be prepared (e.g., trade name “7 Water” (Quasia), etc.).

The non-destructive hydrogen-adding apparatus is a device or instrument that adds hydrogen molecules to a commercially available biologically applicable liquid (e.g., enclosed in a hydrogen-permeable plastic bag, such as a polyethylene bag), such as an intravenous drip, from the outside of the package, and is commercially available from, for example, MiZ Company Limited (http://www.e-miz.co.jp/technology.html). With this apparatus, a bag containing a biologically applicable liquid is immersed in saturated hydrogen water so that the bag is permeated with hydrogen, whereby hydrogen can be aseptically dissolved in the biologically applicable liquid until reaching concentration equilibrium. The apparatus includes, for example, an electrolyzer and a water tank, and water in the water tank circulates between the electrolyzer and the water tank, and can generate hydrogen by electrolysis. Alternatively, a simple disposable instrument can be used for the same purpose (Japanese Patent Laid-Open No. 2016-112562, etc.). This instrument includes a biologically applicable liquid-containing plastic bag (a hydrogen-permeable bag, such as a polyethylene bag) and a hydrogen-generating agent (e.g., metallic calcium, metallic magnesium/cation-exchange resin, etc.) in an aluminum bag, and the hydrogen-generating agent is covered with, for example, a non-woven fabric (e.g., a water vapor-permeable non-woven fabric). Hydrogen generated by wetting the hydrogen-generating agent covered with a non-woven fabric with a small amount of water, such as water vapor, passes through the plastic bag and is non-destructively and aseptically dissolved in the biologically applicable liquid.

The hydrogen gas-containing gas or hydrogen saturated biologically applicable liquid (e.g., sterile water, saline, an intravenous drip, etc.) prepared using the device or instrument described above can be administered orally or parenterally to a patient with sequelae of viral infection.

Another embodiment of the composition of the present invention includes dosage forms (e.g., tablets, capsules, etc.) containing a hydrogen-generating agent that can generate hydrogen in the gastrointestinal tract, prepared to be orally administered to (or ingested by) a patient with sequelae of viral infection. The hydrogen-generating agent is preferably composed of, for example, ingredients approved as foods or food additives.

### 3. Improvement of Symptoms of Sequelae of Viral Infection and/or Suppression of Worsening of Symptoms

A second embodiment of the present invention provides a method for improving symptoms caused by sequelae of viral infection and/or reducing worsening of the symptoms, the method comprising administering the composition of the present invention to a patient with sequelae of viral infection, the symptoms including at least pulmonary fibrosis, feeling of fatigue, malaise, shortness of breath, difficulty breathing, palpitation, muscle weakness, low-grade fever, muscle pain, decreased sensation, joint pain, chest discomfort, chest pain, sore throat, headache, heavy head, body pain, generalized pain, faintness, cognitive impairment, memory impairment, language retrieval difficulty, gastrointestinal disorder, abdominal pain, loss of concentration, dizziness when standing up, fragility, irregular body temperature, chills, hair loss, cold hands and feet, heat and/or cold intolerance, sweating, hot flush, lymph node tenderness, significant changes in body weight, tinnitus, confusion, sleeping disorder, lightheadedness, dizziness, anorexia, feeling of faintness, bradycardia, blood pressure drop, nausea, prolonged cough, brain fog, rash, and/or decrease in activity levels associated with these symptoms.

The composition of the present invention can improve the quality of life (QOL) of patients.

As the method for administering the composition of the present invention to a patient with sequelae of viral infection, when hydrogen gas is used as an active ingredient, for example, pulmonary administration by inhalation, sucking, or the like is preferred. When the gas is inhaled, the gas can be inhaled from the mouth or nose through a nasal cannula or a mask-like instrument that covers the mouth and nose, and the gas can be delivered to the lungs and distributed throughout the body via blood.

When a hydrogen-dissolved liquid is administered to a patient, oral administration, intravenous administration, or intraarterial administration (including infusion) is preferred. The hydrogen-dissolved liquid to be orally administered is preferably stored at low temperatures, and the cooled liquid or the liquid stored at ordinary temperature may be administered to a patient with sequelae of viral infection. Hydrogen is dissolved in water at a concentration of about 1.6 ppm (1.6 mg/L) at ordinary temperature and pressure, and it is known that the difference in solubility due to temperature is relatively small. Alternatively, when the hydrogen-dissolved liquid is in the form of, for example, a hydrogen gas-containing intravenous drip or injection prepared using the non-destructive hydrogen-adding apparatus mentioned above, it may be administered to a patient with sequelae of viral infection by a parenteral administration route, such as intravenous administration or intraarterial administration.

A hydrogen gas-containing gas with the above hydrogen concentration or a hydrogen-dissolved liquid with the above dissolved hydrogen concentration can be administered to a patient with sequelae of viral infection once or several times (e.g., 2 or 3 times) a day over 1 week to 3 months or a longer period of time, for example, 1 week to 6 months or longer. When a hydrogen gas-containing gas is administered, it can be administered per dose over, for example, 10 minutes to 2 hours or longer, preferably 20 minutes to 40 minutes or longer, and more preferably 30 minutes to 2 hours. Further, when a hydrogen gas-containing gas is pulmonary administered by inhalation, sucking, or the like, the gas can be administered to a patient with sequelae of viral infection in an atmospheric pressure environment or, for example, at a high pressure within the range of more than the normal atmospheric pressure (about 1.013 atm) and 7.0 atm or less, for example, in a high-pressure environment within the range of 1.02 to 7.0 atm, preferably 1.02 to 5.0 atm, more preferably 1.02 to 4.0 atm, and further preferably 1.02 to 1.35 atm. Administration in a high-pressure environment can promote the systemic absorption of hydrogen in the patient with sequelae of viral infection.

The high-pressure environment can be created by using a high-pressure housing (e.g., a capsule-like housing) that is designed to have sufficient strength and that can form a high pressure of more than the normal atmospheric pressure and 7.0 atm or less inside, for example, by pressing the hydrogen gas-containing gas (e.g., hydrogen-containing oxygen or air) thereinto. The shape of the high-pressure housing is preferably rounded overall with no corners because it is pressure resistant. Further, the material of the high-pressure housing is preferably lightweight and high-strength, and examples include reinforced plastics, carbon fiber composite materials, titanium alloys, aluminum alloys, and the like. Patients with sequelae of viral infection can undergo administration of a composition containing hydrogen gas together with oxygen gas or air, for use in improving symptoms of sequelae of viral infection and/or reducing worsening of the symptoms, in such a high-pressure housing.

In the treatment of sequelae of viral infection using the composition of the present invention, it is desirable to use a hydrogen gas generator, a hydrogen water generator, or a hydrogen gas-adding device (e.g., the above-mentioned hydrogen gas supply device (or a gaseous hydrogen inhalation device), a hydrogen-adding instrument (or a hydrogen water generator), a non-destructive hydrogen-adding apparatus (a device that non-destructively dissolves hydrogen gas in a biologically applicable liquid, such as an intravenous drip enclosed in a hydrogen-permeable bag), and other devices), for which sufficient therapeutic efficacy and safety have been confirmed.

### EXAMPLES

The present invention will be described in more detail below with reference to Examples; however, the technical scope of the present invention is not limited to these Examples.

### [Example 1] Improvement of Sequelae of Viral Infection and/or Suppression of Worsening of Symptoms by Hydrogen Gas Inhalation

### <Case 1>

### Pathology of Patient

The patient was a Japanese male aged 58 as of 2021. He was infected with the delta variant of coronavirus in 2021. Although the patient was cured after home care, his activity level was lower than that of a healthy person. Fatigue, pain, and decrease in thinking power progressed extremely, and the level of daily life decreased below the level of a healthy person, so he underwent a medical examination at the hospital. The diagnosis based on the physician’s diagnostic criteria was as follows.
(i) Fatigue - persistent, fatigue so pronounced that the activity level is severely impaired: severity 3 (severe)
(ii) Feeling of fatigue after exertion - loss of physical and mental endurance, fatigue aggravated by physical exertion: severity 3 (severe)
(iii) Long recovery time from exertion - it takes 24 hours or more to recover to pre-exertion activity level: severity 2 (moderate)
(iv) Pain - muscle pain, joint pain, headache: severity 2 (moderate)
(v) Sleeping disorder - nonrestorative sleep, insomnia, hypersomnia: severity 2 (moderate)
(vi) Confusion and loss of concentration: severity 2 (moderate)
(vii) Memory impairment - short-term memory is impaired: severity 1 (mild)
(viii) Shortness of breath associated with exertion: severity 2 (moderate)
(ix) Gastrointestinal disorder - diarrhea, irritable bowel syndrome: severity 1 (mild)
(x) Repeated sore throat: severity 1 (mild)
(xi) Dizziness when standing up or fragility: severity 1 (mild)
(xii) Heat/cold intolerance: severity 1 (mild)
(xiii) Body temperature changes, irregular body temperature, cold hands and feet: severity 1 (mild)

### Effect of Hydrogen Gas Inhalation After 2 Weeks of Inhalation

Hydrogen gas inhalation was started from Jul. 7, 2020, using a hydrogen gas inhaler (Jobs-α, hydrogen concentration: about 4% to 5%, 100% hydrogen yield: 200 ml/min) available from MiZ Company Limited (Ofuna, Kamakura-shi). Although the inhalation time was sometimes 3 hours or less due to a cold for a few days after 3 days of hydrogen inhalation, he inhaled hydrogen gas for approximately 3 hours a day.

His head was foggy before hydrogen inhalation; however, after inhalation, his head appeared clear and refreshed. It has been years since he has felt like his brain fog has lifted. The frequency of headache and generalized pain seemed to decrease, and the number of doses of analgesics decreased.

His sympathetic nerves were high before inhalation; however, when he inhaled hydrogen, he felt relaxed and drowsy.

### Effect of Hydrogen Gas Inhalation After 4 Weeks of Inhalation

### (I) Symptoms That Have Been Significantly Recovered Based on The Physician’s Diagnosis Are Itemized as Follows

Fatigue after exertion - loss of physical and mental endurance, fatigue aggravated by physical exertion: before inhalation (severity 3: severe) → after inhalation (severity 2: moderate)
Long recovery time from exertion - it takes 24 hours or more to recover to pre-exertion activity level: before inhalation (severity 2: moderate) → after inhalation (severity 1: mild)
Fatigue - persistent, fatigue so pronounced that the activity level is severely impaired: before inhalation (severity 3: severe) → after inhalation (severity 1: mild, or severity 2: moderate)
Pain, headache: before inhalation (severity 2: moderate) → after inhalation (severity 1: mild)
Loss of concentration: before inhalation (severity 2: moderate) → after inhalation (severity 1: mild)
Repeated sore throat: before inhalation (severity 2: moderate) → after inhalation (severity 1: mild)

Since other symptoms did not worsen, it is considered that worsening thereof was suppressed by hydrogen.

### (II) Patient’s Impressions of Chronic Severe Fatigue

Inhalation of hydrogen gas has significantly alleviated bedridden chronic severe fatigue. Even now, sudden intense fatigue may occur after exertion; however, by inhaling hydrogen for about 90 minutes, he can avoid falling asleep 1 to 2 hours after exertion.

In general, the degree of fatigue varies from day to day. Although he still falls asleep 2 or 3 days a week, he has improved to the extent that he feels better like a healthy person for more than half the days of the week.

### (III) Patient’s Impressions of Body Pain

He used to have headache once or twice a week before inhaling hydrogen. Since the start of hydrogen inhalation, the frequency of headache has decreased significantly, and headache has almost disappeared. After hydrogen inhalation, the number of doses of analgesics decreased to ¼ of that before hydrogen inhalation. Since last year before he started hydrogen gas inhalation, he has been doing muscle training and oxygen exercise on days when his condition is good, which also serves as rehabilitation, in order to recover his physical strength and muscle strength. Before hydrogen gas inhalation, muscle pain and joint pain after training were severe, and it was necessary to take analgesics. Two weeks after starting hydrogen inhalation, those pains began to be greatly alleviated. Even when pain occurred due to training, hydrogen inhalation for about 90 minutes every day almost eliminated the pain the next day. As for body pain, he feels that it is surprisingly effective.

### (IV) Patient’s Impressions of Brain Fog

The patient’s main complaints are cognitive, memory, and concentration deficits; however, the foggy feeling in the brain is alleviated by inhalation of hydrogen gas. He always had to endure and strain himself to do anything before hydrogen gas inhalation, but now, after starting hydrogen inhalation, his head feels much clearer. As for cognitive ability, he feels that he has regained some of his writing and summarizing skills.

Occasionally, after 1 to 2 hours of desk work, a strong fatigue suddenly appears, and symptoms, such as speechlessness and difficulty in comprehending sentences, may appear. Such symptoms were not easily corrected before the start of hydrogen inhalation. However, by the inhalation of hydrogen, such fatigue, as well as body pain, are relieved in a shorter time (2 to 3 hours) than before hydrogen inhalation.

### (V) Summary of Symptoms

Hydrogen is clearly effective for chronic fatigue, pain, and brain fog.

Although there are still strong symptoms of fatigue after exertion and sleeping disorder, fatigue after exertion is often resolved in 2 to 3 hours by hydrogen inhalation. These improvements are astounding results considering that he had been sick for several years. Although sleeping disorder has the same strong symptoms as before, drowsiness after hydrogen inhalation has never been seen before.

For symptoms in which inflammation can be alleviated immediately by hydrogen, it seems to lead to relatively early improvement and/or suppression of worsening of the symptoms.

### <Case 2>

A 43-year-old female who was confirmed by the PCR test to be infected with the omicron variant of coronavirus in 2021 had recovered from pneumonia caused by the coronavirus, and the result of the PCR test was negative; however, sequelae of coronavirus pneumonia remained. Before inhalation of hydrogen gas, she was unable to stand up and maintain standing and sitting postures due to significant impairment of upper and lower limb functions and impairment of trunk functions, and she spent her daytime in bed and used a wheelchair for indoor mobility. Further, she had symptoms, such as generalized pain, difficulty breathing due to pulmonary fibrosis, feeling of faintness, malaise, feeling of fatigue after exertion, sleeping disorder, low-grade fever, bradycardia, and blood pressure drop.

She inhaled hydrogen gas for 6 hours a day from mid-December 2021 (a hydrogen gas inhaler: MiZ Company Limited (Ofuna, Kamakura-shi), Jobs-α, hydrogen concentration: about 4% to 5%, 100% hydrogen yield: 200 ml/min). As a result, her sleeping disorder improved in about 3 days after inhalation, and daily headache and migraine that occurred several times a day were relieved. Even after 3 weeks, hydrogen gas inhalation continues to alleviate symptoms, such as generalized pain, difficulty breathing, feeling of faintness, malaise, feeling of fatigue after exertion, sleeping disorder, low-grade fever, bradycardia, and blood pressure drop, before inhalation of hydrogen gas, and the state of pulmonary fibrosis is improving.

### <Case 3>

A 20-year-old female who was infected with influenza virus type A in December 2020 was cured of influenza; however, she had sequelae, such as shortness of breath, dizziness, malaise, decreased thinking power and motivation, and headache, and she spent most of the day sleeping.

She inhaled hydrogen gas for 5 hours per day from early January 2021 (a hydrogen gas inhaler: MiZ Company Limited (Ofuna, Kamakura-shi), Jobs-α, hydrogen concentration: about 4% to 5%, 100% hydrogen yield: 200 ml/min).

As a result, she felt a little lighter around 3 weeks after inhalation and showed improvement in bowel movements and decreased motivation. Further, nap time decreased. Even after 8 weeks, the number of days without naps increased, and although there was malaise, the amount of time for physical activity increased. She also responded that she felt that she was heading in the right direction. After that, the effect of alleviating symptoms, such as shortness of breath, dizziness, malaise, decreased thinking power and motivation, and headache, was confirmed before inhalation of hydrogen gas.

### INDUSTRIAL APPLICABILITY

The present invention makes it possible to, only by administering hydrogen to a patient with sequelae of viral infection, improve symptoms of sequelae of viral infection and/or suppress worsening of the symptoms, such as pulmonary fibrosis, feeling of fatigue, malaise, shortness of breath, difficulty breathing, palpitation, muscle weakness, low-grade fever, muscle pain, decreased sensation, joint pain, chest discomfort, chest pain, sore throat, headache, heavy head, body pain, generalized pain, faintness, cognitive impairment, memory impairment, language retrieval difficulty, gastrointestinal disorder, abdominal pain, loss of concentration, dizziness when standing up, fragility, irregular body temperature, chills, hair loss, cold hands and feet, heat and/or cold intolerance, sweating, hot flush, lymph node tenderness, significant changes in body weight, tinnitus, confusion, sleeping disorder, lightheadedness, dizziness, anorexia, feeling of faintness, bradycardia, blood pressure drop, nausea, prolonged cough, brain fog, rash, and/or decrease in activity levels associated with these symptoms. Since side effects of hydrogen per se are not known, it can increase the QOL of patients.

What is claimed is:

1. A composition for improving symptoms of sequelae of viral infection and/or reducing worsening of the symptoms in a patient with sequelae of viral infection, comprising a hydrogen gas-containing gas as an active ingredient.

2. The composition of claim 1, wherein the virus of the viral infection is one or more viruses selected from the group consisting of coronavirus, novel coronavirus, influenza virus, norovirus, cytomegalovirus, yellow fever virus, herpes virus, measles virus, rubella virus, chickenpox virus, Ebola virus, Marburg virus, adenovirus, papillomavirus, immunodeficiency virus, hepatitis B virus, hantavirus, and variants of these viruses.

3. The composition of claim 1, wherein the symptoms of sequelae of viral infection include at least pulmonary fibrosis, feeling of fatigue, malaise, shortness of breath, difficulty breathing, palpitation, muscle weakness, low-grade fever, muscle pain, decreased sensation, joint pain, chest discomfort, chest pain, sore throat, headache, heavy head, body pain, generalized pain, faintness, cognitive impairment, memory impairment, language retrieval difficulty, gastrointestinal disorder, abdominal pain, loss of concentration, dizziness when standing up, fragility, irregular body temperature, chills, hair loss, cold hands and feet, heat and/or cold intolerance, sweating, hot flush, lymph node tenderness, significant changes in body weight, tinnitus, confusion, sleeping disorder, lightheadedness, dizziness, anorexia, feeling of faintness, bradycardia, blood pressure drop, nausea, prolonged cough, brain fog, rash, and/or decrease in activity levels associated with these symptoms.

4. The composition of claim 2, wherein the symptoms of sequelae of viral infection include at least pulmonary fibrosis, feeling of fatigue, malaise, shortness of breath, difficulty breathing, palpitation, muscle weakness, low-grade fever, muscle pain, decreased sensation, joint pain, chest discomfort, chest pain, sore throat, headache, heavy head, body pain, generalized pain, faintness, cognitive impairment, memory impairment, language retrieval difficulty, gastrointestinal disorder, abdominal pain, loss of concentration, dizziness when standing up, fragility, irregular body temperature, chills, hair loss, cold hands and feet, heat and/or cold intolerance, sweating, hot flush, lymph node tenderness, significant changes in body weight, tinnitus, confusion, sleeping disorder, lightheadedness, dizziness, anorexia, feeling of faintness, bradycardia, blood pressure drop, nausea, prolonged cough, brain fog, rash, and/or decrease in activity levels associated with these symptoms.

5. The composition of claim 1, wherein the hydrogen gas-containing gas has a hydrogen concentration of more than zero (0) and 18.5 volume% or less.

6. The composition of claim 2, wherein the hydrogen gas-containing gas has a hydrogen concentration of more than zero (0) and 18.5 volume% or less.

7. The composition of claim 3, wherein the hydrogen gas-containing gas has a hydrogen concentration of more than zero (0) and 18.5 volume% or less.

8. The composition of claim 1, wherein the composition is administered by inhalation to the patient.

9. The composition of claim 2, wherein the composition is administered by inhalation to the patient.

10. The composition of claim 3, wherein the composition is administered by inhalation to the patient.

11. A method for producing the composition of claim 1, comprising using a hydrogen gas generator, thereby producing the composition.

12. The method of claim 11, wherein the virus of the viral infection is one or more viruses selected from the group consisting of coronavirus, novel coronavirus, influenza virus, norovirus, cytomegalovirus, yellow fever virus, herpes virus, measles virus, rubella virus, chickenpox virus, Ebola virus, Marburg virus, adenovirus, papillomavirus, immunodeficiency virus, hepatitis B virus, hantavirus, and variants of these viruses.

13. The method of claim 11, wherein the symptoms of sequelae of viral infection include at least pulmonary fibrosis, feeling of fatigue, malaise, shortness of breath, difficulty breathing, palpitation, muscle weakness, low-grade fever, muscle pain, decreased sensation, joint pain, chest discomfort, chest pain, sore throat, headache, heavy head, body pain, generalized pain, faintness, cognitive impairment, memory impairment, language retrieval difficulty, gastrointestinal disorder, abdominal pain, loss of concentration, dizziness when standing up, fragility, irregular body temperature, chills, hair loss, cold hands and feet, heat and/or cold intolerance, sweating, hot flush, lymph node tenderness, significant changes in body weight, tinnitus, confusion, sleeping disorder, lightheadedness, dizziness, anorexia, feeling of faintness, bradycardia, blood pressure drop, nausea, prolonged cough, brain fog, rash, and/or decrease in activity levels associated with these symptoms.
